(19) United States
(12) Patent Application Publication
(10) Pub. No.: US 2022/0287650 A1
(43) Pub. Date: Sep. 15, 2022

(54) HEALTH MONITORING METHOD, ELECTRONIC DEVICE, AND COMPUTER STORAGE MEDIUM

(71) Applicant: HON HAI PRECISION INDUSTRY CO., LTD., New Taipei (TW)

(72) Inventors: HSIANG-LING HSIA, New Taipei (TW); JONG-SHYAN WANG, New Taipei (TW)

(21) Appl. No.: 17/574,515

(22) Filed: Jan. 12, 2022

(30) Foreign Application Priority Data
Mar. 15, 2021 (CN) 202110277792.7

(51) Int. Cl. A61B 5/00; A61B 5/024; A61B 5/11; A63B 24/00

(52) U.S. Cl. CPC A61B 5/7275; A61B 5/742; A61B 5/02416; A61B 5/1118; A63B 24/0062; A63B 2024/0065; A63B 2220/62

(57) ABSTRACT

In a health monitoring method, a photoplethysmography (PPG) signal of a user within a preset time is obtained. Physiological parameters of the user are calculated according to the PPG signal. An age of the user and reference physiological parameters corresponding to the age are also obtained. Differences between the physiological parameters of the user and the reference physiological parameters are calculated. A health status of the user is determined according to the differences. The method improves the efficiency of health monitoring.

### FIELD

The present disclosure relates to data analysis, and particularly to a health monitoring method, an electronic device, and a computer storage medium.

### BACKGROUND

Presently, people pay more attention to personal health, and the demand for timely obtaining personal health data is becoming stronger. People can go to medical institutions (such as a hospital) for physical examination to obtain personal health data. However, the shortage of medical resources and being busy make it impossible for some people to go to medical institutions in time.

### BRIEF DESCRIPTION OF THE DRAWINGS

FIG. 1 shows a flow chart of one embodiment of a health monitoring method of the present disclosure.

FIG. 2 shows an example of a photoplethysmography (PPG) signal.

FIG. 3 illustrates an electronic device for health monitoring according to one embodiment of the present disclosure.

### DETAILED DESCRIPTION

In order to provide a clearer understanding of the objects, features, and advantages of the present disclosure, the same are given with reference to the drawings and specific embodiments. It should be noted that the embodiments in the present disclosure and the features in the embodiments may be combined with each other without conflict.

In the following description, numerous specific details are set forth in order to provide a full understanding of the present disclosure. The present disclosure may be practiced otherwise than as described herein. The following specific embodiments are not to limit the scope of the present disclosure.

Unless defined otherwise, all technical and scientific terms herein have the same meaning as used in the field of the art technology as generally understood. The terms used in the present disclosure are for the purposes of describing particular embodiments and are not intended to limit the present disclosure.

The embodiments of the present disclosure provide a health monitoring method, an electronic device, and a computer-readable storage medium. The method can be applied to the electronic device. The electronic device can be a terminal device or a server. The terminal device can be a smart TV, a mobile phone, a tablet computer, a notebook computer, a desktop computer, a personal digital assistant, or other electronic devices. The server can be a single server or a server cluster composed of multiple servers.

FIG. 1 shows a flow chart of one embodiment of a health monitoring method of the present disclosure. According to different requirements, the illustrated order of blocks is illustrative only and the order of the blocks can be changed. Additional blocks can be added or fewer blocks can be utilized without departing from this disclosure.

At block S11, an electronic device obtains a photoplethysmography (PPG) signal of a user within a preset time.

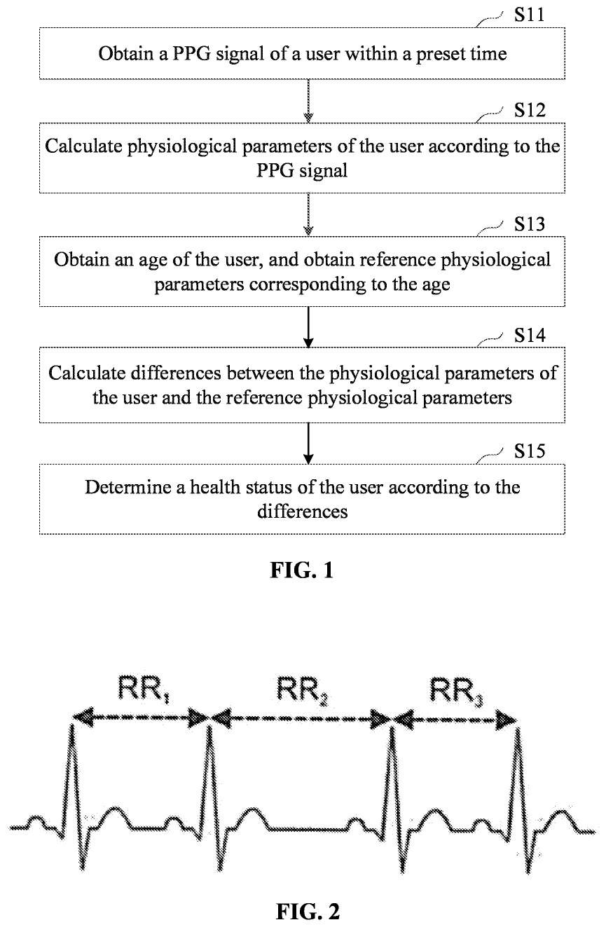

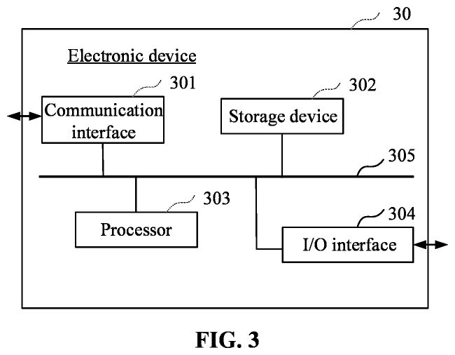

The electronic device can use a PPG sensor (such as a remote photoplethysmography (rPPG) sensor) to obtain the PPG signal. The PPG signal includes multiple cardiac cycles. A cardiac cycle refers to beating of a human heart from the beginning of one heartbeat to the beginning of the next. FIG. 2 shows an example of the PPG signal. Multiple features (such as RR interval (RRI)) are shown in the PPG signal.

The electronic device can be connected to an artificial intelligence (AI) camera equipped with a PPG sensor to obtain the PPG signal.

For example, the preset time is 5 minutes, and the PPG signal is obtained within the 5 minutes. The preset time can be set according to requirements.

In one embodiment, obtaining a PPG signal of a user within a preset time includes: determining whether the user is in a preset area relative to a display screen; controlling the display screen to play a preset video when the user is in the preset area; and obtaining the PPG signal of the user when a playback of the preset video ends.

The display screen can be included in the electronic device or connected to the electronic device. The display screen can be a screen of a TV or a computer monitor. The preset video can be a natural scenery video or a pet animal video. The preset area can be a region where the user can watch the preset video displayed on the display screen. The electronic device can determine whether the user is in the preset area using an infrared sensor.

In some embodiments, when the playback of the preset video ends, the electronic device waits for a preset waiting time, and obtains the PPG signal of the user when the preset waiting time has elapsed. For example, the preset waiting time is 3 minutes, and the preset time is 5 minutes. When the playback of the preset video ends, the electronic device waits for 3 minutes. After 3 minutes have elapsed, the electronic device obtains the PPG signal within the 5 minutes.

In some embodiments, when the playback of the preset video ends, the electronic device determines a viewing time of the user. When the viewing time of the user is longer or equal to a preset viewing time, the electronic device obtains the PPG signal within the preset time.

The viewing time of the user can be determined as a time the user is in the preset area. The preset viewing time can be set according to requirements.

At block S12, the electronic device calculates physiological parameters of the user according to the PPG signal.

In one embodiment, the physiological parameters may include an average heart rate (HR) and a standard deviation of normal-normal intervals (SDNN).

In one embodiment, a formula of calculating the SDNN is:

$$SDNN = \sqrt{\frac{1}{N} {\sum }^{N} {( {RR}_{i} - MeanRR )}^{2}} . \qquad ( formula \text{-} 1 )$$

In the formula-1, RRᵢ is an RRI corresponding to a i-th cardiac cycle. MeanRR is an average RRI of the PPG signal, and N is the number of cardiac cycles of the PPG signal.

A formula of calculating the MeanRR is:

$$MeanRR = \frac{1}{N} {\sum }^{N} {RR}_{i} \qquad ( formula \text{-} 1 )$$

A formula of calculating the average HR is:

MeanHR=60×1000÷Mean RR (formula-3).

In the formula-3, MeanRR is the average HR.

In another embodiment, the physiological parameters may include a heart rate variability (HRV). The HRV may be denoted as an SD1 parameter and an SD2 parameter. The SD1 parameter and the SD2 parameter can be obtained by performing a HRV nonlinear analysis (such as a HRV Poincare Plot analysis) on the PPG signal.

In some embodiments, calculating physiological parameters of the user according to the PPG signal includes: calculating a number of cardiac cycles of the PPG signal; and calculating the physiological parameters of the user according to the PPG signal when the number of the cardiac cycles of the PPG signal is greater than or equal to a preset number.

The number of cardiac cycles of the PPG signal can be the number of RRIs of the PPG signal. For example, the preset number is 180. The electronic device calculates the number of RRIs of the PPG signal, and compares the number of RRIs with the preset number (i.e. 180). If the number of RRIs is equal to or greater than the preset number of 180, the electronic device calculates the physiological parameters of the user according to the PPG signal.

The preset number can be set according to requirements. By setting the preset number and comparing the number of cardiac cycles of the PPG signal with the preset number, the physiological parameters can be calculated more accurately.

In one embodiment, when the number of cardiac cycles is less than the preset number, the electronic device issues a first alert.

The first alert can remind the user that PPG data is insufficient, and also remind the user to seek medical assistance if the user is unwell.

For example, the preset number is 180, the number of RRIs of the PPG signal is calculated, and if the number of RRIs is less than 180, the electronic device issues the first alert. Different information can be included in the first alert. For example, the first time the number of cardiac cycles is less than the preset number, the first alert may be: “Physiological analysis cannot be performed due to excessive variation. If you feel unwell, please consult a doctor.” The second time the number of cardiac cycles is less than the preset number, the first alert may be: “Physiological analysis cannot be performed due to excessive variation. If you feel unwell, please consult your doctor, or a full rest for 2 hours is suggested before retaking the measurement.”

In some embodiment, calculating a number of cardiac cycles of the PPG signal includes: obtaining a feature for each cardiac cycle (nth cardiac cycle) of the PPG signal, the feature being an amplitude feature or a duration feature; calculating a quotient of the feature of the cardiac cycle (nth cardiac cycle) and a corresponding feature of a previous cardiac cycle (n-1th cardiac cycle) of the cardiac cycle; and removing the cardiac cycle (nth cardiac cycle) from the PPG signal when the quotient is greater than a preset quotient. If the quotient is greater than the preset quotient, the cardiac cycle (nth cardiac cycle) can be deemed abnormal.

For example, the preset quotient is 0.3, the feature is RRI. If a quotient of RRI of nth cardiac cycle (RRI(n)) and RRI of n-1th cardiac cycle (RRI(n-1)) is less than 0.3, the nth cardiac cycle is removed from the PPG signal.

The physiological parameters can be calculated more accurately by removing abnormal cardiac cycles from the PPG signal.

At block S13, the electronic device obtains an age of the user, and obtains reference physiological parameters corresponding to the age.

The electronic device can obtain the age of the user using face recognition technology. The electronic device can capture a facial image of the user. and recognize the age of the user according to the facial image.

In one embodiment, the electronic device can obtain a residential area of the user, and obtains the reference physiological parameters according to the age and the residential area of the user.

At block S14, the electronic device calculates differences between the physiological parameters of the user and the reference physiological parameters.

In one embodiment, calculating differences between the physiological parameters of the user and the reference physiological parameters includes: correcting the physiological parameters to obtain corrected physiological parameters; and calculating a difference between the corrected physiological parameters and the reference physiological parameters as the differences.

The physiological parameters of the user can be corrected according to preset rules.

For example, the physiological parameters of the user include SDNN, SD1, and SD2. SDNN, SD1, and SD2 are corrected to obtain corrected SDNN (denoted as TV SDNN), corrected SD1 (denoted as TV SD1), and corrected SD2 (denoted as TV SD2).

A balance parameter can be calculated according to TV SD1 and TV SD2.

In the example, the differences include a difference of SDNN (denoted as SDNN step), a difference of SD1 (denoted as SD1 step), and a difference of SD2 (denoted as SD2 step).

In one embodiment, formulas for calculating the difference of SDNN, the difference of SD1, the difference of SD2 can be defined.

At block S15, the electronic device determines a health status of the user according to the differences.

The differences between the physiological parameters of the user and the reference physiological parameters can reflect the health status of the user. In one embodiment, one or more relationships between differences of physiological parameters and health status are preset, and the electronic device determines the health status of the user according to the one or more relationships.

In one example, the one or more relationships include a first relationship between the difference of SDNN and the health status, a second relationship between the difference of SD1 and the health status, a third relationship between the difference of SD2 and the health status, and a fourth relationship between the balance parameter and the health status.

In some embodiments, the electronic device can calculate a biological age according to the differences, and obtain the health state of the user according to the biological age. The biological age can be correlated to the gender and the age of the user.

When the gender of the user is male and the TV SDNN of the user is greater than or equal to 25, the biological age of the user can be calculated according to a first formula.

When the gender of the user is male and the TV SDNN of the user is less than 25, the biological age of the user can be calculated according to a second formula.

When the gender of the user is female and the TV SDNN of the user is greater than or equal to 25, the biological age of the user can be calculated according to a third formula.

When the gender of the user is female and the TV SDNN of the user is less than 25, the biological age of the user can be calculated according to a fourth formula.

In some embodiments, if the biological age is less than a preset minimum biological age, the electronic device changes the biological age to the preset minimum biological age. If the biological age is greater than a preset maximum biological age, the electronic device changes the biological age to the preset maximum biological age. For example, the preset minimum biological age is 20 and the preset maximum biological age is 90. If the biological age is less than the preset minimum biological age of 20, the electronic device changes the biological age to 20. If the biological age is greater than 90, the electronic device changes the biological age to 90.

in some embodiments, after obtaining the health status of the user, the electronic device sends the health status to a target terminal.

The target terminal is a terminal that can communicate with the user. The target terminal can be a TV, a computer, or a mobile phone. For example, a user A and a user B is making video call. After the health status of the user A is determined, the health status is sent to a terminal of the user B and is displayed on the terminal of the user B.

In some embodiments, the electronic device can further determine an exercise intensity of the user according to the physiological parameters, and determine a target heart rate corresponding to the exercise intensity. If a heart rate of the user is greater than the target heart rate, the electronic device issues a second alert.

A relationship between the physiological parameters and the exercise intensity can be preset. The electronic device determines the exercise intensity of the user according to the physiological parameters of the user and the relationship between the physiological parameters and the exercise intensity.

The target heart rate can be calculated according to a preset formula of heart rate, or be determined according to a relationship between the exercise intensity and the target heart rate.

When the user starts exercising, the electronic device can monitor the heart rate of the user. If the heart rate of the user is greater than the target heart rate, the electronic device issues the second alert.

In some embodiments, the electronic device can further determine a target exercise time of the user according to the health status; obtain an actual exercise time of the user; and calculate a time difference between the actual exercise time and the target exercise time, and issue a third alert.

A relationship between the health state and the target exercise time can be preset, and the target exercise time of the user can be determined according to the relationship between the health state and the target exercise time.

If the time difference is less than a preset time difference, the electronic device can display a message of “You have not completed the exercise target, please continue to exercise.” If the time difference is greater than or equal to the preset time difference, the electronic device can display a message of “Exercise target exceeded, please stop.”

The health monitoring method can improve the efficiency of health monitoring.

FIG. 3 illustrates an electronic device 30 for health monitoring according to one embodiment of the present disclosure. The electronic device 30 can be a server or a terminal device.

The electronic device 30 can be included in a network. The network can include, but is not limited to, the Internet, a wide area network, a metropolitan area network, a local area network, a virtual private network (VPN), etc.

As shown in FIG. 3, the electronic device 30 includes a communication interface 301, a storage device 302, a processor 303, an input/output (I/O) interface 304, and a bus 305. The processor 303 is connected to the communication interface 301, the storage device 302, and the I/O interface 304 through the bus 305.

The communication interface 301 is used for communication. The communication interface 301 may be an existing interface of the electronic device 30 or a newly built interface of the electronic device 30. The communication interface 301 may be a network interface, such as a wireless local area network (WLAN) interface, a cellular network communication interface, or a combination thereof.

The storage device 302 may be used to store program codes and various data of computer programs. For example, the storage device 302 may be used to store a health monitoring system installed in the electronic device 30 and implement completion of storing programs or data during an operation of the electronic device 30. The storage device 302 may include a non-volatile storage medium and a volatile storage medium. The storage device 302 may include a hard disk, a memory, a plug-in hard disk, a smart memory card (SMC), a Secure Digital (SD) card, a flash card, at least one disk storage device, a flash memory device, or other storage medium.

The processor 303 provides computing and control capabilities of the electronic device 30. For example, the processor 303 executes a computer program stored in the storage device 302 to implement the blocks in the health monitoring method.

The processor 303 may be a central processing unit (CPU) or other general-purpose processor, a digital signal processor (DSP), a disclosure specific integrated circuit (ASIC), a field-programmable gate array (FPGA) or other programmable logic device, a discrete gate, or a transistor logic device, or a discrete hardware component, etc. The processor 303 may be a microprocessor or any conventional processor.

The I/O interface 304 is used for input or output data. For example, the I/O interface 304 can be used to connect various input and output devices (mouse, keyboard, three-dimensional touch device, display screen, etc.) to input and output information.

The bus 305 is used to provide a communication channel among the communication interface 301, the storage device 302, the processor 303, and the I/O interface 304 in the electronic device 30.

Those skilled in the art can understand that the structure shown in FIG. 3 is only a representation of a structure related to the present disclosure, and does not constitute a limitation on the electronic device 30 to which the present disclosure is applied. Another example of the electronic device 30 may include more or fewer parts than shown in the figure, or combine some parts, or have a different arrangement of parts.

In one embodiment, the processor 303 executes computer-readable instructions stored in the storage device 302, to implement the operations: obtaining a photoplethysmography (PPG) signal of a user within a preset time; calculating physiological parameters of the user according to the PPG signal; obtaining an age of the user, and obtaining reference physiological parameters corresponding to the age; calculating differences between the physiological parameters of the user and the reference physiological parameters; and determining a health status of the user according to the differences.

Details of the processor 303 executing computer-readable instructions stored in the storage device 302 to implement health monitoring can refer to the description of the flow chart of the health monitoring method, and are not repeated here.

The present disclosure also provides a computer-readable storage medium. The computer-readable storage medium stores a computer program, the computer program includes instructions. The instructions are executed by a processor of an electronic device to implement the health monitoring method.

The computer-readable storage medium may be an internal storage device of the electronic device described above, such as a hard disk or a memory of the electronic device. The computer-readable storage medium may also be an external storage device of the electronic device, such as a plug-in hard disk, a smart memory card (SMC), and a secure digital (SD), a flash card in the electronic device.

The present disclosure is not limited to the above-described exemplary embodiments, and the present disclosure can be embodied in other specific forms without departing from the spirit or essential characteristics of the present disclosure. Therefore, the present embodiments are to be considered as illustrative and not restrictive, and the scope of the present disclosure is defined by the appended claims. All changes and variations in the meaning and scope of equivalent elements are included in the present disclosure. Any reference sign in the claims should not be construed as limiting the claim. Furthermore, the word “comprising” does not exclude other units nor does the singular exclude the plural. A plurality of units or devices stated in the system claims may also be implemented by one unit or one device through software or hardware. Words such as “first” and “second” indicate names, but not in any particular order.

Finally, the above embodiments are only used to illustrate technical solutions of the present disclosure and are not to be taken as restrictions on the technical solutions. Although the present disclosure has been described in detail with reference to the above embodiments, those skilled in the art should understand that the technical solutions described in one embodiment can be modified, or some of the technical features can be equivalently substituted, and that these modifications or substitutions are not to detract from the essence of the technical solutions or from the scope of the technical solutions of the embodiments of the present disclosure.

We claim:

1. A health monitoring method, comprising:
obtaining a photoplethysmography (PPG) signal of a user within a preset time;
calculating physiological parameters of the user according to the PPG signal;
obtaining an age of the user, and obtaining reference physiological parameters corresponding to the age;
calculating differences between the physiological parameters of the user and the reference physiological parameters; and
determining a health status of the user according to the differences.

2. The health monitoring method according to claim 1, wherein calculating physiological parameters of the user according to the PPG signal comprises:
calculating a number of cardiac cycles of the PPG signal; and
calculating the physiological parameters of the user according to the PPG signal when the number of cardiac cycles of the PPG signal is greater than or equal to a preset number.

3. The health monitoring method according to claim 2, wherein calculating a number of cardiac cycles of the PPG signal comprises:
obtaining a feature for each cardiac cycle of the PPG signal, the feature being an amplitude feature or a duration feature;
calculating a quotient of the feature of the cardiac cycle and a corresponding feature of a previous cardiac cycle of the cardiac cycle; and
removing the cardiac cycle from the PPG signal when the quotient is greater than a preset quotient.

4. The health monitoring method according to claim 2, further comprising:
issuing a first alert when the number of cardiac cycles is less than the preset number.

5. The health monitoring method according to claim 1, wherein calculating differences between the physiological parameters of the user and the reference physiological parameters comprises:
correcting the physiological parameters to obtain corrected physiological parameters; and
calculating a difference between the corrected physiological parameters and the reference physiological parameters as the differences.

6. The health monitoring method according to claim 1, wherein obtaining a PPG signal of a user within a preset time comprises:
determining whether the user is in a preset area relative to a display screen;
controlling the display screen to play a preset video when the user is in the preset area; and
obtaining the PPG signal of the user when a playback of the preset video ends.

7. The health monitoring method according to claim 6, further comprising:
determining an exercise intensity of the user according to the physiological parameters, and determining a target heart rate corresponding to the exercise intensity; and
issuing a second alert when a heart rate of the user is greater than the target heart rate.

8. The health monitoring method according to claim 7, further comprising:
determining a target exercise time of the user according to the health status;
determining an actual exercise time of the user; and
calculating a time difference between the actual exercise time and the target exercise time, and issuing a third alert.

9. An electronic device comprising:
at least one processor; and
a storage device storing computer-readable instructions, which when executed by the at least one processor, cause the at least one processor to:
obtain a photoplethysmography (PPG) signal of a user within a preset time;
calculate physiological parameters of the user according to the PPG signal;
obtain an age of the user, and obtain reference physiological parameters corresponding to the age;
calculate differences between the physiological parameters of the user and the reference physiological parameters; and
determine a health status of the user according to the differences.

10. The electronic device according to claim 9, wherein the at least one processor is further caused to:
calculate a number of cardiac cycles of the PPG signal; and
calculate the physiological parameters of the user according to the PPG signal when the number of cardiac cycles of the PPG signal is greater than or equal to a preset number.

11. The electronic device according to claim 10, wherein the at least one processor is further caused to:
obtain a feature for each cardiac cycle of the PPG signal, the feature being an amplitude feature or a duration feature;
calculate a quotient of the feature of the cardiac cycle and a corresponding feature of a previous cardiac cycle of the cardiac cycle; and
remove the cardiac cycle from the PPG signal when the quotient is greater than a preset quotient.

12. The electronic device according to claim 10, wherein the at least one processor is further caused to:
issue a first alert when the number of cardiac cycles is less than the preset number.

13. The electronic device according to claim 9, wherein the at least one processor is further caused to:
correct the physiological parameters to obtain corrected physiological parameters; and
calculate a difference between the corrected physiological parameters and the reference physiological parameters as the differences.

14. The electronic device according to claim 9, wherein the at least one processor is further caused to:
determine whether the user is in a preset area relative to a display screen;
control the display screen to play a preset video when the user is in the preset area; and
obtain the PPG signal of the user when a playback of the preset video ends.

15. A non-transitory storage medium having instructions stored thereon, when the instructions are executed by a processor of an electronic device, the processor is configured to perform a health monitoring method, the method comprising:
obtaining a photoplethysmography (PPG) signal of a user within a preset time;
calculating physiological parameters of the user according to the PPG signal;
obtaining an age of the user, and obtaining reference physiological parameters corresponding to the age;
calculating differences between the physiological parameters of the user and the reference physiological parameters; and
determining a health status of the user according to the differences.

16. The non-transitory storage medium according to claim 15, wherein calculating physiological parameters of the user according to the PPG signal comprises:
calculating a number of cardiac cycles of the PPG signal; and
calculating the physiological parameters of the user according to the PPG signal when the number of cardiac cycles of the PPG signal is greater than or equal to a preset number.

17. The non-transitory storage medium according to claim 16, wherein calculating a number of cardiac cycles of the PPG signal comprises:
obtaining a feature for each cardiac cycle of the PPG signal, the feature being an amplitude feature or a duration feature;
calculating a quotient of the feature of the cardiac cycle and a corresponding feature of a previous cardiac cycle of the cardiac cycle; and
removing the cardiac cycle from the PPG signal when the quotient is greater than a preset quotient.

18. The non-transitory storage medium according to claim 16, wherein the method further comprising:
issuing a first alert when the number of cardiac cycles is less than the preset number.

19. The non-transitory storage medium according to claim 15, wherein calculating differences between the physiological parameters of the user and the reference physiological parameters comprises:
correcting the physiological parameters to obtain corrected physiological parameters; and
calculating a difference between the corrected physiological parameters and the reference physiological parameters as the differences.

20. The non-transitory storage medium according to claim 15, wherein obtaining a PPG signal of a user within a preset time comprises:
determining whether the user is its a preset area relative to a display screen;
controlling the display screen to play a preset video when the user is in the preset area; and
obtaining the PPG signal of the user when a playback of the preset video ends.
